(19) United States
(12) Patent Application Publication
(10) Pub. No.: US 2025/0009739 A1
(43) Pub. Date: Jan. 9, 2025

(54) METHOD OF TREATING A SKIN DISORDER

(71) Applicant: PHAMRI Norden AB, Nynäshamn (SE)

(72) Inventors: Rebecca Szafran, Lidingö (SE); Mårten Winge, Sunnyvale, CA (US); Ilija Batljan, Nynäshamn (SE)

(73) Assignee: PHAMRI Norden AB, Nynäshamn (SE)

(21) Appl. No.: 18/274,078

(22) PCT Filed: Jan. 25, 2022
(86) PCT No.: PCT/SE2022/050071

§ 371 Date: Jul. 25, 2023

(30) Foreign Application Priority Data
Jan. 25, 2021 (SE) 2150076-4
Mar. 10, 2021 (SE) 2150271-1

(51) Int. Cl. A61K 31/4965; A61P 17/00

(52) U.S. Cl. CPC A61K 31/4965; A61P 17/00

(57) ABSTRACT

The present invention relates to a method of treating a skin disorder in a subject in need thereof by administering to the human an effective dose of a compound represented by formula (I)

### FIELD OF THE INVENTION

The present invention relates to the field of pharmaceutical treatment, and in particular treatment of disorders of the skin with compounds capable of inhibiting ion channel exchangers ENaC, NHE and NCX.

### BACKGROUND

The skin is a semi-permeable barrier that is central for maintaining homeostasis, and preventing excess water loss, microbial assaults, thermoregulation and sensation. To maintain these functions, the skin requires close regulation of its function involving proliferation, differentiation and epidermal immune interactions. Ion channel flux is central for regulating these functions, and cations such as sodium and calcium are tightly regulated both extra- and intracellularly by ion channel exchangers. Changes in intracellular Na and membrane potential can modulate the activity of basolateral Na/H and Na/Ca exchangers which will affect this cross-talk (Harvey et al., 1995). This means that an alteration in one of these channels lead to compensatory mechanisms in others. An example of this is where regulation of all individual conductance need not occur in the correct direction in the homeostatic sense, provided a sufficient subset of remaining conductance are appropriately regulated (O'Leary et al., 2013).

The regulation of these channels is complex, however it is widely accepted that, for many different electrically excitable cells, voltage-gated calcium channels provide an indirect measure of membrane voltage changes, and that the subsequent influx of calcium ions can trigger changes in ion channel expression. A pharmacologic demonstration of this phenomenon is that treatment with the sodium channel blocker mexiletine results in upregulation of sodium channel transcription and expression, and treatment with the calcium channel blocker verapamil produces a similar effect to that seen with sodium channel blockade, suggesting that the response to sodium channel blockade is mediated by changes in calcium ion fluxes (Rosati et al, 2004).

Another example is how sodium overload can prompt NCX overactivity while sodium depletion can also promote NCX1 mediated responses and production of proinflammatory cytokine release from immune cells (Harvey et al., 1995).

WO9009792 suggests amiloride and some derivatives thereof for treatment of inflammatory skin and eye disorders.

WO2015168574 suggests the use of epithelial ion channel (ENaC) blockers, such as benzamil, for the treatment of psoriasis.

WO2020150606 suggests a method for treating a disease of immune dysregulation in a subject, said method comprising administering to the subject a therapeutically effective amount of e.g. benzamil. The disease of immune dysregulation may be an inflammatory disease, which in turn may be a dermatological disorder.

### SUMMARY

The present invention describes the use of specific amiloride derivatives that has a combined off target profile that has a directionality that mimics benzamil, with activity on all three ion channels ENaC, NHE1 and NCX1. Such amiloride derivatives are distinct and differential in their effect on skin inflammation compared to specific ENaC inhibitors with reduced or no activity on NHE and NCX.

The present invention utilizes combined inhibition of multiple ion channels (ENaC, NHE and NCX) by amiloride-derived drugs to enable restoration of aberrant signaling by cytokines involved in skin inflammation, where expression or activity of several of these channels is dysregulated, enabling these drugs as treatment for a variety of disorders of the skin. Amiloride derivatives useful in the present invention are those of formula (I)

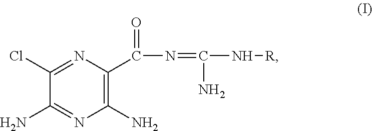

wherein R is selected from
—C(CH₃)CH₂C(CH₃)₃;

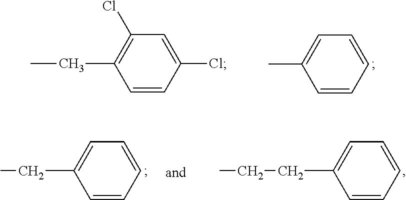

and pharmaceutically acceptable salts thereof.

According to a first aspect there is provided a method of treating a skin disorder in a subject in need thereof by administering to the human an effective dose of a compound represented by formula (I)

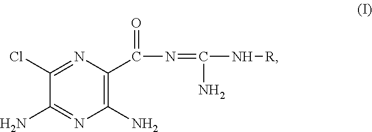

wherein R is selected from
—C(CH₃)CH₂C(CH₃)₃;

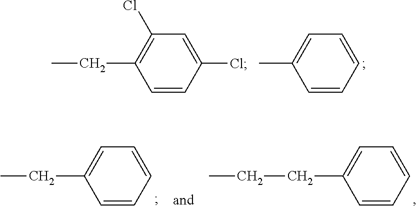

and pharmaceutically acceptable salts thereof,
with the proviso that when the compound is phenamil or benzamil then the skin disorder is not psoriasis.

The disorder of the skin may be selected from the group where either CXCL1, TNFα and/or IL1α are deregulated, e.g. where abnormal levels of CXCL1, TNFα and/or IL1a are present and implicated in the pathogenesis, and/or consisting of acne, atopic dermatitis, seborrheic dermatitis, nummular dermatitis, periorificial dermatitis, rosacea, vitiligo, Hidradenitis suppurativa, lupus, morphea, scleroderma, cutaneous ulcers, inflamed seborrheic keratoses, nevi, fibrous papules, Pityriasis rubra pilaris, Pemphigus, including pemphigus vulgaris and pemphigus vegetans, Bullous pemphigoid, IgA pemphigus, Keratosis follicularis, Lamellar ichthyosis, Epidermolytic ichthyosis, Netherton's syndrome, congenital ichthyosiform erythroderma, Cutaneous vasculitis, Behçet's disease, Cutaneous graft versus host disease, SAPHO (synovitis, acne, pustulosis, hyperostosis and osteitis) syndrome, Sarcoidosis, Panniculitis, Stevens Johnson syndrome, Toxic epidermal necrolysis, Neutrophilic dermatoses, pyoderma gangrenosum, Sweet Syndrome, Sneddon Wilkinson, Acute generalized exanthematous pustulosis (AGEP), dermatitis herpetiformis, Dermatomyositis, Cryoporin-associated periodic syndromes, Familial mediterranean Fever, Xeroderma pigmentosum, Pruritus, pain, UV-induced skin damage, irritant contact dermatitis, Still's disease, Lichen planus, eczema, wound, lesion, ulcer, alopecia, as well as cutaneous neoplasms with inflammation including actinic keratosis, squamous cell carcinoma in situ, squamous cell carcinoma, basal cell carcinoma, extramammary Paget's disease, dermatofibroma protuberans, atypical fibroxanthoma, sebaceous carcinoma; and Merkel cell carcinoma.

The compound represented by formula (I) may be administered topically or systemically.

The compound may be injected or given orally.

The subject being treated may be a mammalian subject, such as a human or animal.

When the compound is in the form of a pharmaceutically acceptable salt, the salt may be selected from the lactic acid salt, acetic acid salt, and phosphoric acid salt.

In some embodiments, the compound is administered in an amount effective to achieve a serum concentration of 5-50 ng/ml in the subject.

According to a second aspect there is provided a compound represented by formula (I)

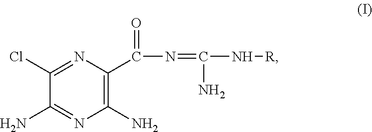

wherein R is selected from
—C(CH₃)CH₂C(CH₃)₃;

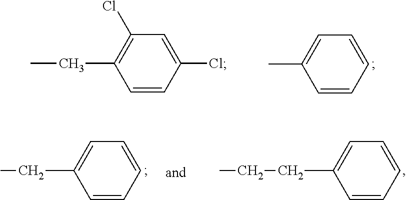

and pharmaceutically acceptable salts thereof, for use in a method of treatment described above.

According to a third aspect there is provided a use of a compound represented by formula (I)

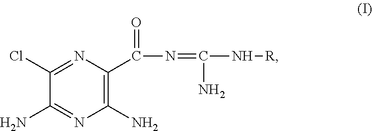

wherein R is selected from
—C(CH₃)CH₂C(CH₃)₃;

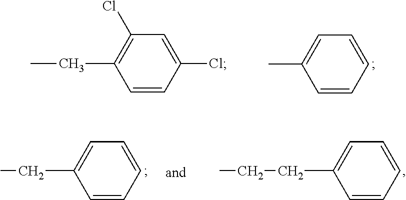

and pharmaceutically acceptable salts thereof, in the manufacture of a pharmaceutical composition for use in a method of treatment described above.

### BRIEF DESCRIPTION OF THE FIGURES

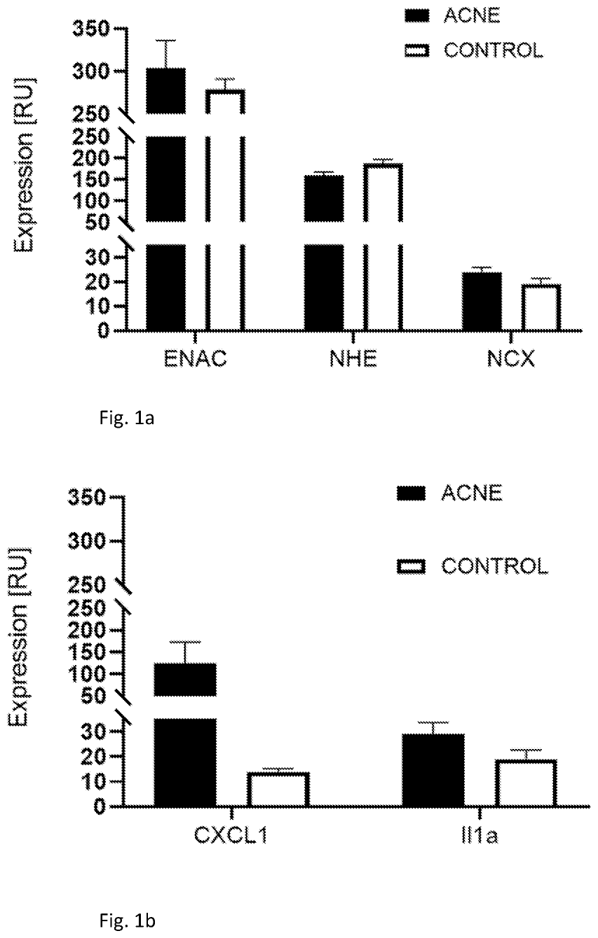

In FIG. 1a is shown that in patients with acne, ENaC and NCX1 are upregulated and NHE is downregulated compared to control persons without acne.

In FIG. 1b it is shown that CXCL1 and IL1a are upregulated in patients with acne.

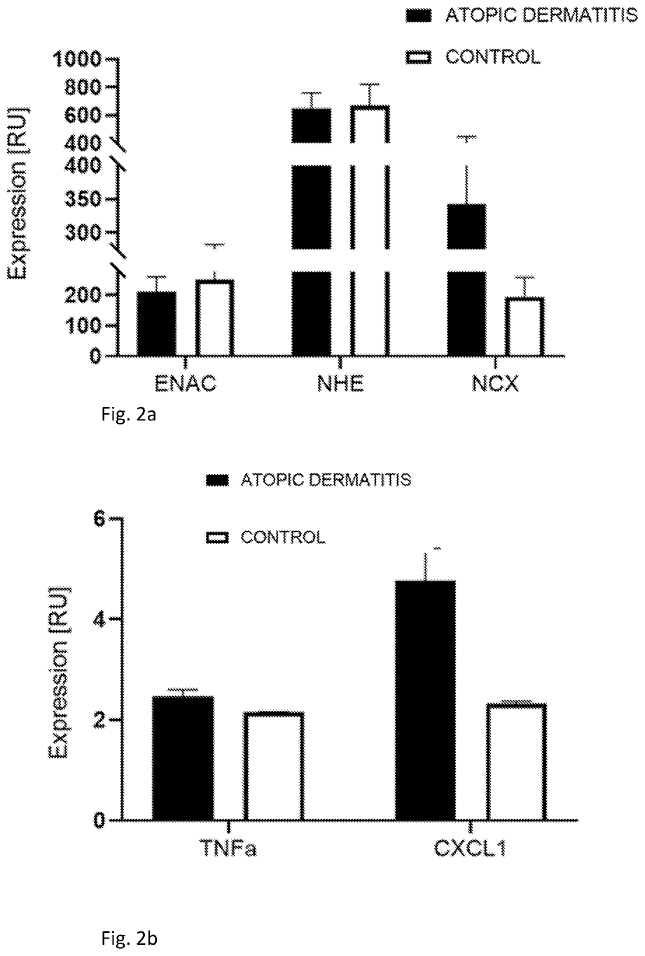

In FIG. 2a is shown that in patients with atopic dermatitis, NCX1 is upregulated and ENAC is downregulated compared to control persons without atopic dermatitis.

In FIG. 2b it is shown that CXCL1 and TNFα are upregulated in patients with eczema.

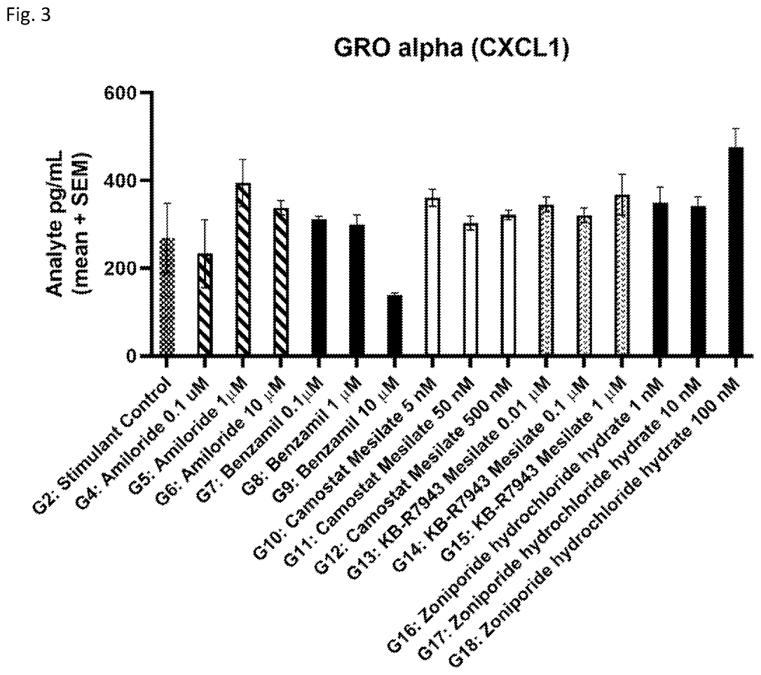

In FIG. 3 is shown that benzamil reduces expression of CXCL1 in stimulated keratinocytes.

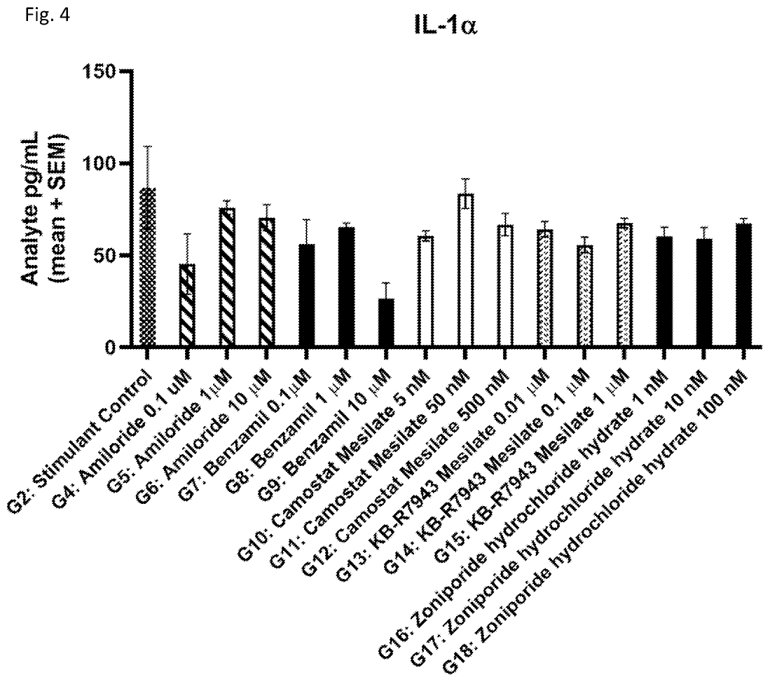

In FIG. 4 is shown that benzamil reduces expression of IL1a in stimulated keratinocytes.

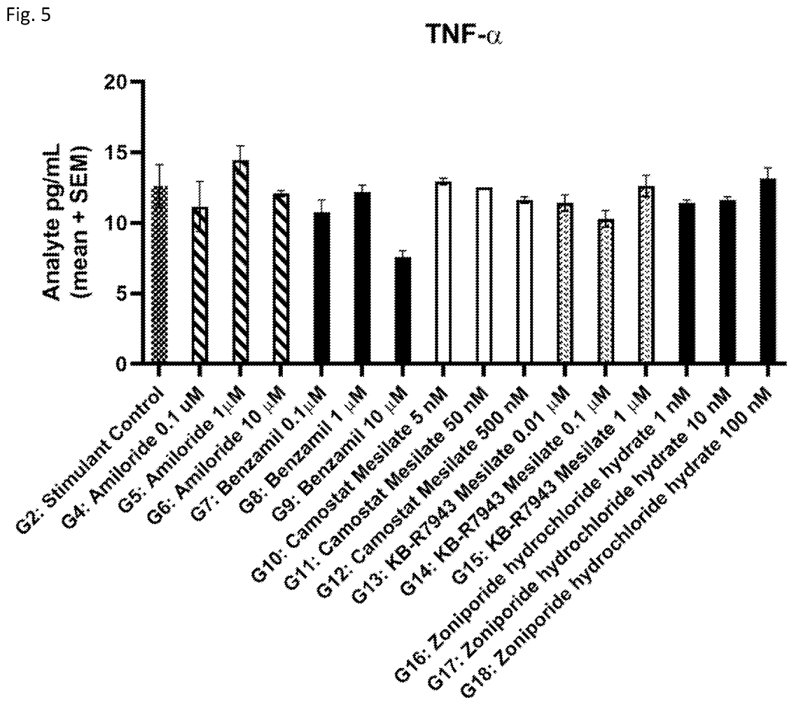

In FIG. 5 is shown that benzamil reduces expression of TNFα in stimulated keratinocytes.

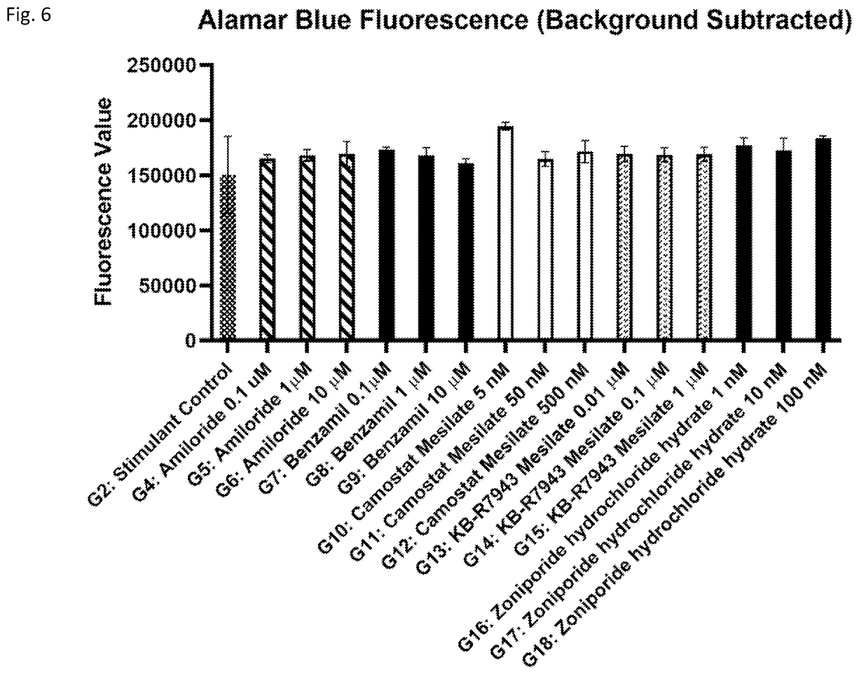

In FIG. 6 is shown that benzamil does not reduce proliferation in low-level inflamed keratinocyte conditions.

### DETAILED DESCRIPTION

The amiloride derivative benzamil has been suggested for use in treatment of psoriasis (WO2015168574, A1) and cystic fibrosis (Hirsh et al., 2004). Although benzamil is a more potent ENaC inhibitor in vitro than amiloride it has previously been shown that the in vivo efficacy of benzamil and phenamil specifically on ENaC is equivalent to amiloride (Hirsh et al., 2004). It would thus be expected that a higher dose of amiloride would give a partial or similar effect should ENaC be the specific target underlying the effect on keratinocyte proliferation.

Loss of ENaC, NCX1 or NHE individually does not alter intracellular ion channel signaling to the extent of when multiple channels are inhibited (Rosati et al, 2004). It has been demonstrated that differences in expression of NCX1 (Staiano et al., 2009), NHE1 and ENaC correlate with activation, and they may compensate for each other.

The present inventors have discovered that it is the distinct off-target profile of benzamil as compared to amiloride or specific sodium channel inhibitors that enables benzamil's therapeutic effect, and inhibits proinflammatory cytokine production in skin cells underlying inflammation in inflammatory skin disease through combined ENaC, NCX1 and NHE inhibition.

The present invention thus relates to therapeutic methods using compounds that shares the properties of combined ENaC, NCX1 and NHE inhibition with benzamil, for treatment of conditions caused or complicated by proinflammatory cytokine production in skin cells with the proviso that when the compound is phenamil or benzamil then the skin disorder is not psoriasis. In some embodiments, when the compound is benzamil, the skin disorder is not atopic dermatitis, alopecia areata, bulloid pemphigus, chronic eczema, dermatomyositis, erythema nodosum, epidermolysis bullosa, hydradenitis suppurativa, lichen planus, pemphigus vulgaris, pyoderma gangrenosum, scleroderma, or vitiligo.

The experimental results disclosed herein demonstrate that compounds with a combined inhibitory effect of all three ion channels ENaC, NCX1 and NHE in combination, exemplified by benzamil, is sufficient and necessary to prevent proinflammatory signaling through the inflammatory cytokines CXCL1, TNFα and IL1a.

A number of amiloride derivatives with a combined inhibitory effect of all three ion channels ENaC, NCX1 and NHE in combination are known in the art and include compound represented by formula (I)

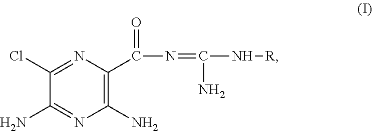

wherein R is selected from
—C(CH₃)CH₂C(CH₃)₃;

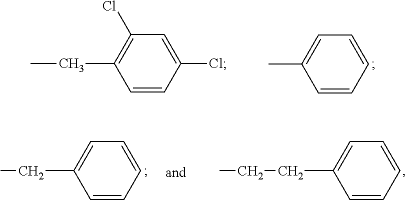

### (Kleyman et al., 1988).

The specific amiloride derivatives disclosed above (also termed “compounds of the invention” herein) have similar profiles targeting all three ion channels ENaC, NHE and NCX1, and therefore have therapeutic potential in treatment of disorders of the skin with combined inflammation and aberrant keratinocyte proliferation, as described herein.

The inflammatory cytokines CXCL1, TNFα and/or IL1a have been proven to underlie the pathogenesis of skin disorders where aberrant epidermal signaling lead to an inflammatory immune response. These conditions include acne (Li, X., et al., 2019), atopic dermatitis (Farley, S. M., et al., 2006) (He, H., et al., 2021), seborrheic dermatitis (Molinero, L. L., et al., Clin Immunol), nummular dermatitis (Farley, S. M., et al., 2006), periorificial dermatitis (Farley, S. M., et al., 2006), rosacea (Li, N., et al., 2014), vitiligo (Jimbo, H., et al., 2020), Hidradenitis suppurativa (Gupta and Skinner, 2004), lupus (Norman, R., et al., 2006), morphea (Fett, N., 2013), scleroderma, cutaneous ulcers, inflamed seborrheic keratoses, nevi, fibrous papules, Pityriasis rubra pilaris (Muller, H., et al., 2008), Pemphigus, including pemphigus vulgaris and Pemphigus vegetans (Tavakolpour, S., et al., 2020), Bullous pemphigoid (Tabatabaei-Panah, P. S., et al., 2019), IgA pemphigus (Howell, S. M., et al., 2005), Keratosis follicularis (Mayuzumi, N., et al., 2005), Lamellar ichthyosis (Malik, K., et al., 2019), Epidermolytic ichthyosis (Malik, K., et al., 2019), Netherton's syndrome (Malik, K., et al., 2019), congenital ichthyosiform erythroderma (Malik, K., et al., 2019), Cutaneous vasculitis (Carbone, F. and F. Montecucco, 2015), Behçet's disease (van der Houwen and van Laar, 2020), Cutaneous graft versus host disease (Gupta and Skinner, 2004), SAPHO (synovitis, acne, pustulosis, hyperostosis and osteitis) syndrome (Gupta and Skinner, 2004), Sarcoidosis (Gupta and Skinner, 2004), Panniculitis (Gupta and Skinner, 2004), Stevens Johnson syndrome (Wang, C. W., et al., 2018), Toxic epidermal necrolysis (Gupta and Skinner, 2004), Neutrophilic dermatoses (Ahn, C. et al., 2018), pyoderma gangrenosum (Ahn, C. et al., 2018), Sweet Syndrome (Gupta and Skinner, 2004), Sneddon Wilkinson (Gupta and Skinner, 2004), Acute generalized exanthematous pustulosis (AGEP) (Feldmeyer, L., et al., 2016), dermatitis herpetiformis (Amerio, P., et al., 2000), Dermatomyositis (Alexis and Strober, 2005), Cryoporin-associated periodic syndromes (Luo, X. Y., et al., 2020), Familial mediterranean Fever (Samuels and Ozen, 2006), Xeroderma pigmentosum (Kunisada, M., et al., 2017), Pruritus (Deftu, A. F., et al., 2018), pain (Deftu, A. F., et al., 2018), UV-induced skin damage (Fukunaga, A., et al., 2021), irritant contact dermatitis (Funch, A. B., et al., 2021), Still's disease (Kadavath, S. and P. Efthimiou, 2015), Lichen planus (Wu, T., et al., 2013), eczema (Farley, S. M., et al., 2006), wound (Zheng, R., et al., 2019), lesion, ulcer, alopecia (Loh, S. H., et al., 2018), as well as cutaneous neoplasms with inflammation including actinic keratosis (Maru, G. B., et al., 2014), squamous cell carcinoma in situ (Maru, G. B., et al., 2014), squamous cell carcinoma, basal cell carcinoma (Maru, G. B., et al., 2014), extramammary Paget's disease, dermatofibroma protuberans, atypical fibroxanthoma, sebaceous carcinoma; and Merkel cell carcinoma.

The present amiloride derivatives activate and/or alter the immune response, which in turn affects the carcinomas listed above.

Skin disorders wherein CXCL1 is deregulated include acne, Xeroderma pigmentosum, Pruritus, Pain, irritant contact dermatitis, Lichen planus, atopic dermatitis.

Skin disorders wherein TNFα is deregulated include Pityriasis rubra pilaris, Pemphigus, including pemphigus vulgaris and pemphigus vegetans, Bullous pemphigoid, IgA pemphigus, Keratosis follicularis, Lamellar ichthyosis, Epidermolytic ichthyosis, Netherton's syndrome, congenital ichthyosiform erythroderma, Cutaneous vasculitis, Behcet's disease, Cutaneous graft versus host disease, SAPHO (synovitis, acne, pustulosis, hyperostosis and osteitis) syndrome, Sarcoidosis, Panniculitis, Stevens Johnson syndrome, Toxic epidermal necrolysis, Neutrophilic dermatoses, pyoderma gangrenosum, Sweet Syndrome, Sneddon Wilkinson, AGEP, dermatitis herpetiformis, Scleroderma, Morphea, Dermatomyositis, Cryoporin-associated periodic syndromes, Familial mediterranean Fever, UV-induced skin damage, Still's disease, hidradenitis suppurativa, alopecia, vitiligo, basal cell carcinoma, actinic keratosis, and squamous cell carcinoma including squamous cell carcinoma in situ (Bowen's disease).

Skin disorders wherein IL1a is deregulated include Bullous pemphigoid, Keratosis follicularis, Cutaneous vasculitis, Cryoporin-associated periodic syndrome, Familial mediterranean Fever, UV-induced skin damage, Still's disease, Lichen planus, wounds, seborrheic dermatitis, and alopecia.

A composition comprising an effective dose of benzamil and compounds or salts with a similar combined inhibitory effect on all of ENaC, NCX1 and NHE, optionally combined with additional therapeutic agents, may be provided to an individual suffering from any of the above listed conditions. The administration can preferably be per oral or topical, etc. In some embodiments topical is preferred. The dosing and periodicity of administration is selected to provide for therapeutic efficacy.

The present invention thus relates to and makes use of pharmaceutically acceptable compositions which comprise a therapeutically-effective amount of at least one compound according to the invention, optionally in the form of a pharmaceutically acceptable salt, optionally combined with one or more additional agents for treatment of the relevant skin disorders, formulated together with one or more pharmaceutically acceptable excipients. The active ingredients and excipient(s) may be formulated into compositions and dosage forms according to methods known in the art. The pharmaceutical compositions of the present invention may be formulated for administration in solid, liquid or semi-liquid form, including those adapted for the following: oral administration, for example, tablets, capsules, powders, granules, pastes for application to the tongue, aqueous or non-aqueous solutions or suspensions, drenches, or syrups; or topical application, for example, as a lotion, cream, ointment, spray, patch, microneedle array, etc. applied to the skin. The medicaments, pharmaceutical compositions or therapeutic combinations according to the present invention may be in any form suitable for the application to humans and/or animals, preferably humans including infants, children and adults and can be produced by standard procedures known to those skilled in the art. The medicament, (pharmaceutical) composition or therapeutic combination can be produced by standard procedures known to those skilled in the art, e.g. from “Pharmaceutics: The Science of Dosage Forms”, Second Edition, Aulton, M.E. (ED. Churchill Livingstone, Edinburgh (2002); “Encyclopedia of Pharmaceutical Technology”, Second Edition, Swarbrick, J. and Boylan J.C. (Eds.), Marcel Dekker, Inc. New York (2002); “Modern Pharmaceutics”, Fourth Edition, Banker G.S. and Rhodes C.T. (Eds.) Marcel Dekker, Inc. New York 2002, “The Theory and Practice of Industrial Pharmacy”, Lachman L., Lieberman H. And Kanig J. (Eds.), Lea & Febiger, Philadelphia (1986).

An effective dose of benzamil and compounds or salts with a similar combined inhibitory effect of both ENaC, NCX1 and NHE may include a “therapeutically effective dose or amount” or a “prophylactically effective dose or amount”. A “therapeutically effective dose/amount” refers to an amount effective, at dosages and for periods of time necessary, to achieve the desired therapeutic result. A therapeutically effective amount may vary according to factors such as the disease state, age, sex, and weight of the individual, and the ability to elicit a desired response in the individual. A therapeutically effective dose/amount is also one in which any toxic or detrimental effects are outweighed by the therapeutically beneficial effects. A “prophylactically effective dose/amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

It has been found in animal studies by the present inventors that a serum concentration of about 20 nanogram/mL (62.5 nM) is therapeutically effective. Thus, in one embodiment, the compounds of the invention are provided to a subject in a dose sufficient to achieve a serum concentration of about 20 ng/ml, such as 5-50 ng/ml. It is currently assessed that a serum concentration of 5-50 ng/ml of a compound of the invention corresponds to a dose of about 0.1-20 mg to be administered to a human subject. As comparison, human oral dosing of the sodium channel inhibitor amiloride requires 10 mg for a serum concentration of 20.6 ng/ml in adults (Jones et al., 1997). The dose to be administered depends on the route of administration, age and body mass of the subject, and the bioavailability of the active ingredient to be administered, which in turn may be influenced by the dosage form used, as is known in the art (Adajare, 2020). Methods for the assessment of topical drug bioavailability are known in the art, e.g. (Herkenne et al., 2008).

Dosage regimens may be adjusted to provide the optimum desired response (e.g., a therapeutic or prophylactic response). For example, a single dose may be administered, several divided doses may be administered over time or the dose may be proportionally reduced or increased as indicated by the exigencies of the therapeutic situation. The dose may be administered to the subject upon symptoms of skin disease, or before onset of symptoms.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

As used herein, the term “dose amount” refers to the quantity, e.g., milligrams (mg), of the substance which is administered to the subject. In one embodiment, the dose amount is a fixed dose, e.g., is not dependent on the weight of the subject to which the substance is administered. In another embodiment, the dose amount is not a fixed dose, e.g., is dependent on the weight of the subject to which the substance is administered, or for a topical therapy a dose may be related to the surface area that is treated, e.g. dose/m² of skin.

Exemplary dose amounts, e.g., fixed dose amounts, for use in treating an adult human may include, about 0.01 mg, about 0.05 mg, about 0.1 mg, about 0.5 mg, about 1 mg, about 5 mg, about 10 mg, about 20 mg, about 50 mg, about 100 mg, about 500 mg, or more.

Exemplary dose amounts, e.g., dose amounts for topical use treating an adult human by the methods of the invention include, about 0.01 mg/m² surface area, about 0.05 mg/m² surface area, about 0.1 mg/m² surface area, about 0.5 mg/m² surface area, about 1 mg/m² surface area, about 5 mg/m² surface area, about 10 mg/m² surface area, about 20 mg/m² surface area, about 50 mg/m² surface area, about 100 mg/m² surface area, about 500 mg/m² surface area, or more.

Ranges intermediate to the above-recited ranges are also contemplated. For example, ranges having any one of these values as the upper or lower limits are also intended to be part of the invention, e.g., about 0.01 mg to about 100 mg, about 1 mg to about 10 mg, etc.

The administration of the composition may comprise a recurring cycle of administration of composition to the subject. The periodicity of administration of the compound may be about once a week, once every other week, about once every three weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks, about once every 21 weeks, about once every 22 weeks, about once every 23 weeks, about once every 24 weeks, about once every 5-10 days, about once every 10-20 days, about once every 10-50 days, about once every 10-100 days, about once every 10-200 days, about once every 25-35 days, about once every 20-50 days, about once every 20-100 days, about once every 20-200 days, about once every 30-50 days, about once every 30-90 days, about once every 30-100 days, about once every 30-200 days, about once every 50-150 days, about once every 50-200 days, about once every 60-180 days, or about once every 80-100 days. Periodicities intermediate to the above-recited times are also contemplated by the invention. Ranges intermediate to the above-recited ranges are also contemplated by the invention. For example, ranges having any one of these values as the upper or lower limits are also intended to be part of the invention, e.g., about 110 days to about 170 days, about 160 days to about 220 days, etc.

“Duration of a periodicity” refers to a time over which the recurring cycle of administration occurs. For example, a duration of the periodicity of administration of a substance may be may be up to about 4 weeks, up to about 8 weeks, up to about 12 weeks, up to about 16 weeks or more, up to about 20 weeks, up to about 24 weeks, up to about 28 week, up to about 32 weeks or more, during which the periodicity of administration is about once every week. For example, a duration of the periodicity may be about 6 weeks during which the periodicity of administration is about once every 4 weeks, e.g., the substance is administered at week zero and at week four.

The compounds of the invention can be provided in the form of a pharmaceutically acceptable salt. As used herein, “pharmaceutically acceptable salts” refer to derivatives of the disclosed compounds wherein the parent compound is modified by making acid or base salts thereof.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isethionic, and the like. Presently preferred pharmaceutically acceptable salts are prepared from lactic, acetic, and phosphoric acid, which have been found to have improved properties at least in terms of improved stability, increased water solubility, and/or reduced polymorphism (as described in co-pending patent application EP21199548.5). The pharmaceutically acceptable salts of compounds of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington: The Science And Practice Of Pharmacy (Adajare, 2020).

All references cited herein are incorporated by reference in their entirety. The examples below are included to illustrate the invention and shall not be considered as limiting the scope of the invention, which is as defined in the appended claims.

### EXPERIMENTAL

By in silico experimentation we have found that several of the ion channels ENaC, NCX1 and NHE, rather than individual ion channels, are deregulated (up or downregulated) in conditions with skin inflammation, as exemplified in skin disorders with an abnormal cytokine signaling. In FIG. 1a is shown that ENaC and NCX1 are upregulated and NHE is downregulated in persons with acne as compared to control persons without acne. In FIG. 1b it is shown that the cytokines CXCL1 and IL1a are upregulated in patients with acne as compared to control persons without acne.

In FIG. 2a is shown that in patients with atopic dermatitis NCX1 is upregulated and ENAC is downregulated compared to control persons without atopic dermatitis. In FIG. 2b it is shown that the cytokines CXCL1 and TNFα are upregulated in patients with atopic dermatitis.

Primary keratinocytes were stimulated with TNFα (5 ng/ml) for 24 hours and expression of pro-inflammatory cytokines involved in inflammatory skin disease was analyzed with benzamil as inhibitor and compared to inhibitors with different affinity to ENaC/NCX1/NHE1 (amiloride), ENaC but not NCX1 and NHE1 (Camostat mesilate), NCX1 but not ENaC or NHE1 (KB-R7943) or NHE but not ENaC or NCX1 (zoniporide).

Primary human keratinocytes were thawed according to manufacturer's instructions, washed in growth medium (GM) and viability assessed via Tryphan blue. Cells were incubated at 37° C. with 5% CO₂. After confluence, a stock cell solution of 1.5×10⁵ cells/ml was prepared in GM. 200 μL of the cell suspension was added to each well of respective 96 well plates for a total of 3×10⁴ cells/well. Cells were incubated at 37° C. with 5% CO₂ overnight for adherence. After overnight incubation, cell culture media was removed from each well and 100 μL of fresh GM was added to each well. Appropriate concentrations of inhibitor compound, vehicle and TNFα was prepared in GM of a final volume of 3 ml. Concentrations of non-benzamil inhibitors were chosen to be 0.1×, 1× and 10× the IC50 for each primary target. For Amiloride 0.1, 1 and 10 um (IC50 for ENaC 100 nM), for Camostat Mesilate 5 nm, 50 nm and 500 nM (IC50 50 nM for ENaC), for KB-R7943 0.01 uM, 0.1 uM and 1 uM (IC50 5-10 uM for NCX), Zoniporide 1 nM, 10 nM and 100 nM (IC50 14 nM for NHE). Concentration of TNF alpha was selected to recapitulate a low level of pro-inflammatory cytokine stimulation.

100 μl of each treatment was added to appropriate wells. Samples were incubated for 24 hours. 20 hours post TNFα stimulation, 20 μL of alamar blue was added for each well. Cells were incubated for 4 hours at 37° C. 24 hours post TNFα stimulation, the fluorescence of each well was read at 544/590 nm for cell viability. The supernatants were collected and stored at −80° C. for cytokine analysis. The inflammatory cytokines TNFα, CXCL1 and IL1a in the cell culture supernatants were analysed by Luiminex. Data was exported into Excel and processed using Graphpad Prism 9.1.

### Results

Benzamil reduce expression of CXCL1 (FIG. 3), IL1a (FIG. 4) and TNFα (FIG. 5) distinct from amiloride, Camostat Mesilate (extracellular ENaC inhibitor with different off-target profile), KB-R7943 (NCX1 inhibitor) or Zoniporide (NHE inhibitor), in a dose-dependent manner. It was found that a concentration above 1 uM, where ENaC, NCX1 and NHE1 were predicted to be targeted, was efficacious, and complete inhibition of ENAC but low or no inhibition of NCX1 or NHE1 are not sufficient.

Benzamil did not reduce proliferation under these conditions (FIG. 6), which indicates that the reduced expression of inflammatory cytokines was not due to a toxic effect. These results demonstrate that the combined inhibitory effect on all three of ENAC, NCX1 and NHE (NOT one or another) exhibited by benzamil is sufficient and necessary to prevent proinflammatory signaling. CXCL1, TNFα and IL1a have been proven to be important for the pathogenesis of skin inflammation (see e.g. FIGS. 1 and 2).

### REFERENCES

Adajare, A. (Ed.). (2020). Remington: The Science And Practice Of Pharmacy (23 ed.). Academic Press.
Ahn, C. et al. (2018). Pyoderma gangrenosum: a review of pathogenesis and treatment. Expert Rev Clin Immunol, 14 (3), 225-233.
Alexis and Strober. (2005). Off-label dermatologic uses of anti-TNF-α therapies. J Cutan Med Surg, 9 (6), 296-302.
Amerio, P., et al. (2000). Expression of eotaxin, interleukin 13 and tumour necrosis factor-alpha in dermatitis herpetiformis. Br J Dermatol, 143 (5), 974-978.
Carbone, F. and F. Montecucco. (2015). Inflammation in arterial diseases. IUBMB Life, 67 (1), 18-28. Deftu, A. F., et al. (2018). CXCL1 activates TRPV1 via Gi/o protein and actin filaments. Life Sci, 193, 282-291.
Farley, S. M., et al . . . (2006). Fas ligand elicits a caspase-independent proinflammatory response in human keratinocytes: implications for dermatitis. J Invest Dermatol, 126 (11), 2438-2451.
Feldmeyer, L., et al. (2016). Acute Generalized Exanthematous Pustulosis: Pathogenesis, Genetic Background, Clinical Variants and Therapy. Int J Mol Sci, 17 (8).
Fett, N. (2013). Scleroderma: nomenclature, etiology, pathogenesis, prognosis, and treatments: facts and controversies. Clin Dermatol, 31 (4), 432-437.
Fukunaga, A., et al. (2021). Bioactive substances in the stratum corneum of the epidermis found as indicators of skin damage due to sun exposure. Photodermatol Photoimmunol Photomed.
Funch, A. B., et al. (2021). CD8(+) tissue-resident memory T cells recruit neutrophils that are essential for flare-ups in contact dermatitis. Allergy.
Gupta and Skinner. (2004). A review of the use of infliximab to manage cutaneous dermatoses. J Cutan Med Surg, 8 (2), 77-89.
Harvey et al. (1995). Cross-talk between sodium and potassium channels in tight epithelia. Kidney International, 48, 1191-199.
He, H., et al. (2021). Mild atopic dermatitis lacks systemic inflammation and shows reduced nonlesional skin abnormalities. J Allergy Clin Immunol, 147 (4), 1369-1380.
Herkenne et al. (2008). In Vivo Methods for the Assessment of Topical Drug Bioavailability. 25 (1), 87-103.
Hirsh et al. (2004). Evaluation of Second Generation Amiloride Analogs as Therapy for Cystic Fibrosis Lung Disease. THE JOURNAL OF PHARMACOLOGY AND EXPERIMENTAL THERAPEUTICS, 311, 929-938.
Howell, S. M., et al. (2005). Rapid response of IgA pemphigus of the subcorneal pustular dermatosis subtype to treatment with adalimumab and mycophenolate mofetil. J Am Acad Dermatol, 53 (3), 541-543.
Jimbo, H., et al. (2020). Fas-FasL interaction in cytotoxic T cell-mediated vitiligo: The role of lesional expression of tumor necrosis factor-alpha and interferon-gamma in Fas-mediated melanocyte apoptosis. Exp Dermatol, 29 (1), 61-70.
Jones et al. (1997). Pharmacokinetics of Amiloride after Inhalation and Oral Administration in Adolescents and Adults with Cystic Fibrosis. Pharmacotherapy, 17 (2), 263-270.
Kadavath, S. and P. Efthimiou. (2015). Adult-onset Still's disease-pathogenesis, clinical manifestations, and new treatment options. aNN mED, 47 (1), 6-14.
Kleyman et al. (1988). Amiloride and Its Analogs as Tools in the Study of Ion Transport. J Membran Biol., 105, 1-21.
Kunisada, M., et al. (2017). CXCL1 Inhibition Regulates UVB-Induced Skin Inflammation and Tumorigenesis in Xpa-Deficient Mice. J Invest Dermatol, 137 (9), 1975-1983.
Li, N., et al. (2014). Alarmin function of cathelicidin antimicrobial peptide LL37 through IL-36gamma induction in human epidermal keratinocytes. J Immunol, 193 (10), 5140-5148.
Li, X., et al. (2019). Identification of Genes and Pathways Associated with Acne Using Integrated Bioinformatics Methods. Dermatology, 235 (6), 445-455.
Loh, S. H., et al. (2018). Role of T helper 17 cells and T regulatory cells in alopecia areata: comparison of lesion and serum cytokine between controls and patients. J Eur Acad Dermatol Venereol, 32 (6), 1028-1033.
Luo, X. Y., et al. (2020). Tumor necrosis factor-alpha blockade ameliorates inflammatory response in two children with chronic infantile neurological, cutaneous and articular syndrome. J Dermatol, 47 (8), 903-906.
Malik, K., et al. (2019). Ichthyosis molecular fingerprinting shows profound TH17 skewing and a unique barrier genomic signature. J Allergy Clin Immunol, 143 (2), 604-618.
Maru, G. B., et al. (2014). The role of inflammation in skin cancer. Adv Exp Med Biol, 816, 437-469.
Mayuzumi, N., et al. (2005). Effects of ultraviolet B irradiation, proinflammatory cytokines and raised extracellular calcium concentration on the expression of ATP2A2 and ATP2C1. Br J Dermatol, 152 (4), 697-701.
Molinero, L. L., et al. (Clin Immunol). Up-regulated expression of MICA and proinflammatory cytokines in skin biopsies from patients with seborrhoeic dermatitis. 106 (1), 50-54.
Muller, H., et al. (2008). Infliximab monotherapy as first-line treatment for adult-onset Pityriasis rubra pilaris: case report and review of the literature on biologic therapy. J Am Acad Dermatol, 59 (5 Suppl), S65-70.
Norman, R., et al. (2006). Case reports of etanercept in inflammatory dermatoses. J Am Acad Dermatol, 54 (3 Suppl 2), S139-142.
O'Leary et al. (2013). Correlations in ion channel expression emerge from homeostatic tuning rules. Proceedings of the National Academy of Sciences, 110 (28), E2645-E265.
Rosati et al. (2004). Regulation of Ion Channel Expression. Circ Res., 94, 874-883.
Samuels and Ozen. (2006). Familial Mediterranean fever and the other autoinflammatory syndromes: evaluation of the patient with recurrent fever. Curr Opin Rheumatol, 18 (1), 108-117.
Staiano et al. (2009). Expression and function of Na1/Ca exchangers 1 and 3 in human macrophages and monocytes. Eur. J. Immunol., 39, 1405-1418.
Tabatabaei-Panah, P. S., et al. (2019). Proinflammatory Cytokine Gene Polymorphisms in Bullous Pemphigoid. Front Immunol, 10, 636.
Tavakolpour, S., et al. (2020). Pathogenic and protective roles of cytokines in pemphigus: A systematic review. Cytokine, 129, 155026.
van der Houwen and van Laar. (2020). Behcet's Disease, and the Role of TNF-alpha and TNF-alpha Blockers. Int J Mol Sci, 21 (9).
Wang, C. W., et al. (2018). Randomized, controlled trial of TNF-alpha antagonist in CTL-mediated severe cutaneous adverse reactions. J Clin Invest, 128 (3), 985-996.
Wu, T., et al. (2013). IL-1 alpha regulates CXCL1, CXCL10 and ICAM1 in network form in oral keratinocytes. Clin Lab, 59 (9-10), 1105-1111.
Zheng, R., et al. (2019). Keratinocyte Integrin alpha3beta1 Promotes Secretion of IL-1alpha to Effect Paracrine Regulation of Fibroblast Gene Expression and Differentiation. J Invest Dermatol, 139 (9), 2029-2038 e2023.

What is claimed is:

1. A method of treating a skin disorder in a subject in need, wherein the method comprises administering a therapeutically effective dose of a compound represented by formula (I) 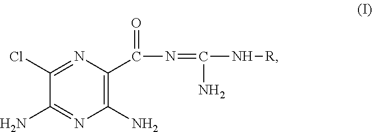
wherein R is selected from
—C(CH₃)CH₂C(CH₃)₃; 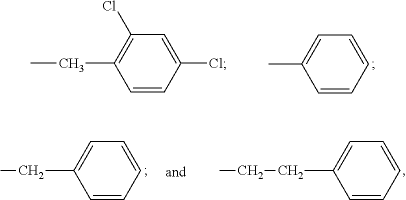
or a pharmaceutically acceptable salt thereof,
wherein when the compound is phenamil or benzamil the skin disorder is not psoriasis.

2. The method according to claim 1, wherein the skin disorder is selected from a group of skin disorders wherein one or more of CXCL1, TNFα and IL1α are deregulated.

3. The method according to claim 1 or 2, wherein the disorder of the skin disorder is selected from a group consisting of acne, atopic dermatitis, seborrheic dermatitis, nummular dermatitis, periorificial dermatitis, rosacea, vitiligo, Hidradenitis suppurativa, lupus, morphea, scleroderma, cutaneous ulcers, inflamed seborrheic keratoses, nevi, fibrous papules, Pityriasis rubra pilaris, Pemphigus, including Pemphigus vulgaris and Pemphigus vegetans, Bullous pemphigoid, IgA pemphigus, Keratosis follicularis, Lamellar ichthyosis, Epidermolytic ichthyosis, Netherton's syndrome, congenital ichthyosiform erythroderma, Cutaneous vasculitis, Behçet's disease, Cutaneous graft versus host disease, SAPHO (synovitis, acne, pustulosis, hyperostosis and osteitis) syndrome, Sarcoidosis, Panniculitis, Stevens Johnson syndrome, Toxic epidermal necrolysis, Neutrophilic dermatoses, pyoderma gangrenosum, Sweet Syndrome, Sneddon Wilkinson, Acute generalized exanthematous pustulosis (AGEP), dermatitis herpetiformis, Dermatomyositis, Cryoporin-associated periodic syndromes, Familial mediterranean Fever, Xeroderma pigmentosum, Pruritus, pain, UV-induced skin damage, irritant contact dermatitis, Still's disease, Lichen planus, eczema, wound, lesion, ulcer, alopecia, as well as cutaneous neoplasms with inflammation including actinic keratosis, squamous cell carcinoma in situ, squamous cell carcinoma, basal cell carcinoma, extramammary Paget's disease, dermatofibroma protuberans, atypical fibroxanthoma, sebaceous carcinoma; and Merkel cell carcinoma.

4. The method according to claim 1, wherein the compound represented by formula (I), or a pharmaceutically acceptable salt thereof, is administered topically or systemically.

5. The method according to claim 1, wherein the subject is a human or non-human mammalian subject.

6. The method according to claim 5, wherein the compound represented by formula (I), or a pharmaceutically acceptable salt thereof, is administered topically or systemically to the subject.

7. The method according to claim 1, wherein a pharmaceutically acceptable salt of the compound represented by formula (I) is administered to the subject.

8. The method according to claim 1, wherein the compound represented by formula (I) is administered in an amount effective to achieve a serum concentration of 5-50 ng/ml in the subject.

9. A pharmaceutical composition comprising a therapeutically effective dose of a compound is represented by formula (I) 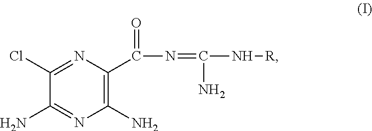
wherein R is selected from
—C(CH₃)CH₂C(CH₃)₃; 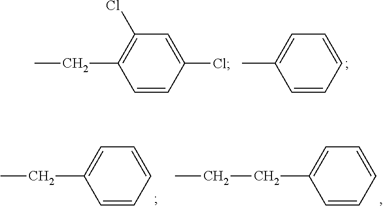
or pharmaceutically acceptable salt thereof.

10. A pharmaceutical composition comprising a therapeutically effective dose of a compound represented by formula (I) 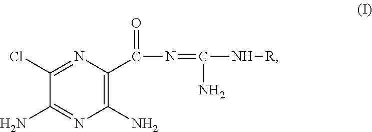
wherein R is selected from
—C(CH₃)CH₂C(CH₃)₃; 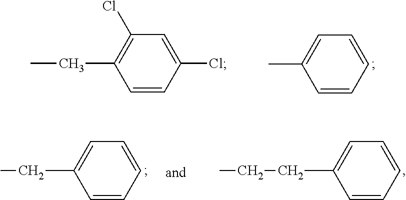
or a pharmaceutically acceptable salt thereof.
